(19) United States
(12) Patent Application Publication
(10) Pub. No.: US 2019/0225931 A1
(43) Pub. Date: Jul. 25, 2019

(54) Algae Beads

(71) Applicant: Algae to Omega Holdings, Inc., Oakland Park, FL (US)

(72) Inventor: Geronimos DIMITRELOS, Plantation, FL (US)

(21) Appl. No.: 16/252,116

(22) Filed: Jan. 18, 2019

Related U.S. Application Data
(60) Provisional application No. 62/619,423, filed on Jan. 19, 2018.

(51) Int. Cl. C12N 1/12

(52) U.S. Cl. CPC C12N 1/12; C12N 2533/30; C12N 2533/74; C12N 2531/00; C12N 2533/18; C12N 2537/10; C12N 2527/00; C12N 2533/76

(57) ABSTRACT

Algae-containing beads comprising a microorganism such as unicellular microalgae, an insoluble carbon source such as char, water, and a crosslinked organic matrix. The beads can further contain clay, such as kaolin.

### CROSS-REFERENCE TO RELATED APPLICATIONS

This application claims the benefit of U.S. Provisional Application 62/619,423, filed Jan. 19, 2018. The entire disclosure of the above application is incorporated herein by reference.

### BACKGROUND

Algae are a group of diverse organisms including microalgae and macroalgae which resemble plants but lack true leaves, stems, roots and vascular tissue/systems. They are mainly characterized by their ability to carry out photosynthesis to provide all or a part of the carbon they require for growth (being primarily phototrophic, although some algae are capable of heterotrophic or mixotrophic growth based on their evolutionary mechanisms available). Algae can evolve with changes in environments and environmental conditions.

Since algae take up carbon dioxide in the process of photosynthesis, they are implicated in carbon sequestration, and can be a renewable carbon source for a variety of foodstuffs, organic compounds, and other carbon-based materials. For example, algae may be used as feedstock for animals such as fish, chickens, and cattle. They may also be a source of naturally-derived antioxidants and other materials useful in cosmetic, nutraceutical and pharmaceutical products. They are most notably known as a biofuel replacement for oil while entrenching themselves in the solar, battery and hydrogen cell industries. Among the most commercially used microalgae are Chlorella and Spirulina. In addition, Dunaliella, Haematococcus, Crypthecodinium, Schizochytrium, Scenedesmus, Aphanizomenon, Arthrospira, Odontella, Isochrysis, Nannochloropsis, Tetraselmis, Phaeodactylum, and Porphyridium are gaining acceptance in animal feed, functional food, pharmaceutical, cosmeceutical, nutraceutical, and industrial material products.

Microalgae, in particular, are amenable to commercial production, such as using photobioreactors in dedicated facilities. However, they are notoriously challenging to grow in facilities on an industrial scale because their growth depends on precision in providing the proper amount and balance of nutrients, carbon sources, light, and temperature. Human intervention is required during cultivation to ensure that the algae do not “crash” during their initial growth phase or while their growth is reaching the exponential stage. Especially at a scale necessary for feeding the world's supply of fish and other animals, algae cultivation remains a challenge. Accordingly, there is a need for improved methods of algae production, particularly for commercial scale production of algae for use in producing food and other products.

### SUMMARY

The current technology provides algae-containing beads and methods for their manufacture. The beads contain a microorganism such as unicellular microalgae, an insoluble carbon source such as char, water, and a crosslinked organic matrix. The beads can further contain clay, such as kaolin.

The nature of the crosslinked organic matrix is such that the organic matrix before crosslinking contains a plurality of hydroxyl groups that react with a divalent cation to crosslink the matrix. Suitable polymers used in various embodiments of the current teachings include water soluble polysaccharides, such as sodium alginate, the class of galactomannans, gellan gum, carrageenan, and agarose. Divalent cations of the alkaline earth metals may be used to cros slink the organic matrix to form the beads. In a particular embodiment, the crosslinking cation is provided in the form of calcium chloride.

In various embodiments, the insoluble carbon source is a char such as a biochar. Biochar may be produced from a suitable biomass, such as algae, rice hulls, and other forms of biomass.

The beads preferably further contain cations and anions corresponding to the nutrients required to grow a strain of algae before it is incorporated into the algal beads of the current teachings. These nutrients include soluble salts of cations such as those selected from Na+, K+, Mg+2, Ca+2, Fe+3, Mn+2, Zn+2, Cu+2, and Co+2, as well as soluble salts of one or more anions which include those selected from nitrates, phosphates, hydrogen phosphates, dihydrogen phosphates, sulfates, chlorides, EDTA, carbonates, bicarbonates, and molybdates. In various embodiments, beads include divalent ions selected from those of Group II of the periodic table (the alkaline earth metals).

The present technology also provides methods of making algae-containing pellets. In various embodiments, such methods involve first making a char suspension by combining water, a unicellular microorganism such as the microalgae further described herein, an insoluble carbon source such as a char, and a soluble polymer. The soluble polymer comprises a plurality of hydroxyl groups that can react with a suitable cross-linking agent, such as a source of divalent cations, to crosslink the polymer. The char suspension is then divided into bead volumes or globules using methods described herein. The bead shaped globules are then contacted with a solution of a divalent cation to gel the soluble polymer and form beads containing the microorganisms and the insoluble carbon source (e.g. microalgae and char, including biochar). In various embodiments, the solution contains divalent alkaline earth metal cations, selected from Be+2, Mg+2, Ca+2, Sr+2, and Ba+2. The polymer containing a plurality of hydroxyl groups in various embodiments is selected from polyvinyl alcohol, water soluble polysaccharides, alginate, agarose, carrageenan, galactomannans, gellan gum, guar gum, and so on without limitation.

In various embodiments, the char suspension includes a biochar, such as biochar produced from algal biomass or any plant biomass material such as rice hulls. In various embodiments, the char suspension further contains a clay or other aluminosilicate material. In these and other embodiments described herein, the char suspension can further comprise any additive that is on a list of components generally recognized as safe.

In various embodiments, the char suspension is divided into bead globules that are crosslinked to form the beads. One way of doing this is to flow the char suspension in a conduit from a proximal to a distal end of the conduit, wherein the distal end of the conduit comprises a restriction that causes droplets to form upon exit from the conduit. The nature of the restriction and the velocity of flow through the conduit are selected to provide that the char suspension exiting the conduit is in the form of beads or droplets, such as those produced at the end of a syringe tip. In various embodiments, the conduit in which the char suspension is flowing contains at least one vertical passage before the exit from the conduit on its distal end. Beads formed from the droplets exiting the conduit in these embodiments exhibit an advantageous diversity of densities, as later described.

Methods of growing algae in culture comprise adding the beads to a medium and placing the seeds in contact with and in motion relative to the medium. This can be accomplished in any conventional way, such as by stirring the medium while the medium is in contact with the beads or by rocking a vessel that contains both the beads and the medium.

### DRAWINGS

FIGS. 1 and 2 are diagrams illustrating methods of forming beads according to the present technology.

FIGS. 3 and 4 are diagrams illustrating aspects of the geometry of the beads according to the present technology.

FIG. 5 is a schematic of a system used to mold beads according to the present technology.

It should be noted that the figures set forth herein are intended to exemplify the general characteristics of materials and methods among those of the present technology, for the purpose of the description of certain embodiments. These figures may not precisely reflect the characteristics of any given embodiment, and are not necessarily intended to define or limit specific embodiments within the scope of this technology.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

### Algae Beads

The present technology provides algae-containing pellets, or “beads,” comprising microalgae and insoluble carbon, in a polymer matrix. In various embodiments, the present technology provides algal beads comprising unicellular microalgae, char, and a crosslinked organic matrix.

In various embodiments, beads are solid or semi-solid pellets comprising algae and a polymer matrix. Without limitation, the beads may have a diameter of from about 1 mm to about 2 cm in diameter, or from about 2 mm to about 9 mm, or from about 3 mm to about 6 mm in diameter. The beads may have any three-dimensional shape, but in many embodiments are spheroidal. As referred to herein a “spheroidal” pellet or bead may be truly spherical, essentially spherical, or spheroidal, and may have irregular features (such as described below regarding flat, convex or concave surface features). In some embodiments, the beads are polyhedral, such as resembling an icosahedron. The surface may be smooth (including substantially smooth surfaces having minor irregularities), rough, or having ribs or other repeating or random surface structures. Without limiting the scope, mechanism, function or utility of present technology, it is believed that beads having such irregular surface features facilitate greater mixing of beads in a bioreactor vessel relative to spheroidal beads, by one or more of altering their motion through culture media, disrupting the boundary layer effect of liquid media on the algae, and reducing adherence of algae to surfaces within a bioreactor. For example, beads having irregular features may spin as they travel through media or bounce in a random fashion as they impact the walls of a bioreactor vessel.

In various embodiments, beads may be opaque, transparent, or translucent, and may be reflective in some embodiments. In various embodiments, beads have transmissive or reflective properties such that (without limiting the scope, mechanism, function or utility of the present technology) light deflects, reflects, and passes through the beads.

In various embodiments, algae are entrained in the polymer matrix, such that the algae are both exposed to the components of the beads (e.g., biochar or other insoluble carbon source and, optionally, nutrients) and exposed to the culture media or other liquid into which the pellets are introduced during use. In various embodiments, the beads comprise surface features that allow penetration of liquid into the interior of the beads, such as pores, fissures, or other openings. Without limiting the scope, mechanism, function or utility of present technology, such beads provide a locus for algae growth, effectively seeding or inoculating a liquid medium for algae growth in methods of the present technology, wherein algae grow and are released from the beads into the liquid medium. The beads may also be a source of other components to the algal growth medium, including char, clay, and other nutrients which may be released from the beads into the medium, providing additional nutrients for the algae to grow. It should be noted that, in various embodiments, this feature of the present technology is in contrast with algae-containing constructs among those known in the art, which entrain algae but do not allow growth or release of algae into a medium, such as for use in removing pollutants or other materials from the medium. In particular, unlike immobilized algae of the prior art, the beads described herein can be used to inoculate a growth medium with cells that can be harvested after a suitable growth period.

As discussed further herein, beads are used for the production of algae, which may be further used in the production of food stuffs, cosmetics, nutraceuticals, pharmaceuticals and other products for human or animal consumption, as well as for biofuels and production of other carbon-based materials. Accordingly, the materials used in the compositions and methods of the present technology are preferably biocompatible. Such “biocompatible” materials are, preferably, non-toxic to the microorganisms to be grown with the beads in which the materials are used, at the levels at which the materials are used. In embodiments wherein the algae are used for production of materials to be consumed or used nutritionally, cosmetically, or therapeutically by humans or other animals (for example, in or as food stuffs, cosmetics, nutraceuticals, and pharmaceuticals), biocompatible materials are safe for use with humans or other animals, for the intended purpose. In various embodiments, the materials are generally recognized as safe (“GRAS”), according to applicable food, drug or cosmetic regulatory standards, such as those issued by the United States Food & Drug Administration. In various embodiments, the beads consist of materials suitable for human or animal consumption.

### Microalgae

In various embodiments the beads of the present technology comprise algae, in particular, microalgae. As the term is used in the current teachings, algae refer to unicellular organisms (e.g., protists) that are, or are hybridized or otherwise evolved from, unicellular phototropic organisms (i.e., capable of carrying out photosynthesis). In some embodiments, such algae are heterotrophic, or mixotrophic. Such unicellular organisms may be distinguished from more complex organisms such as seaweeds that are also known as algae, and more specifically, macroalgae.

The term microalgae embraces species described as cyanobacteria, as well as green, red, and brown algae. Microalgae among those useful herein include Eugelenophyta, Chyrisphyta, Pyrrophyta, Chlorophyta, Rhodophyta, and Xanaophyta. Genera of algae useful herein include Aphanizomenon, Arthrospira, Chlorella, Crypthecodinium, Dunaliella, Euglena, Haematococcus, Isochrysis, Nannochloropsis, Odontella, Phaeodactylum, Picochlorum, Porphyridium, Scenedesmus, Schizochytrium, Spirulina, and Tetraselmis.

Aphanizomenon is a cyanobacterium with a rather short history of human consumption. Aphanizomenon flos-aquae has many beneficial health effects such as anti-inflammatory, exhaustion relief, assisting digestion, and general improvement of overall wellbeing. Aphanizomenon are technically classified as bacteria but share properties with bacteria and with plants. They contain many biologically active substances that have beneficial effects on human health thus a large research interest in the use of blue-green algae for food supplementation has emerged. Blue-green algae are among the most primitive living organisms on Earth and several blue-green algae have pronounced antibacterial properties. Aphanizomenon species among those useful herein include Aphanizomenon americanum, Aphanizomenon aphanizomenoides, Aphanizomenon balticum, Aphanizomenon capricorni, Aphanizomenon chinense, Aphanizomenon cyaneum, Aphanizomenon elenkinii, Aphanizomenon favaloroi, Aphanizomenon flexuosum, Aphanizomenon flos-aquae, Aphanizomenon gracile, Aphanizomenon holsaticum, Aphanizomenon holtsaticum, Aphanizomenon hungaricum, Aphanizomenon incurvum, Aphanizomenon is satschenkoi, Aphanizomenon kaufmannii, Aphanizomenon klebahnii, Aphanizomenon manguinii, Aphanizomenon morrenii, Aphanizomenon ovalisporum, Aphanizomenon paraflexuosum, Aphanizomenon platense, Aphanizomenon schindleri, Aphanizomenon skujae, Aphanizomenon slovenicum, Aphanizomenon sphaericum, Aphanizomenon strictum, Aphanizomenon tropicalis, Aphanizomenon ussaczevii, Aphanizomenon volzii, and Aphanizomenon yezoense.

Arthrospira is a genus of free-floating filamentous cyanobacteria characterized by cylindrical, multicellular trichomes in an open left-hand helix. A dietary supplement made from A. platensis and A. maxima is known as spirulina. (The A. maxima and A. platensis species were once classified in the genus Spirulina.) Although the introduction of two separate genera (Arthrospira and Spirulina) is now generally accepted, there has been much dispute in the past and the resulting taxonomical confusion is tremendous. Spirulina is widely known as a food supplement today, but there are a variety of other possible applications for this cyanobacterium. As an example, it is suggested to be used medically for patients for whom it is difficult to chew or swallow food, or as a natural and cheap drug delivery carrier. Further, promising results in the treatment of certain cancers, allergies and anemia, as well as hepato-toxicity and vascular diseases were found. Next to that, spirulina would also be interesting as a healthy additional animal feed, especially if the price of its production can be further reduced. Spirulina may also be used in technical applications, such as the biosynthesis of silver nanoparticles, which allows the formation of metallic silver in an environmentally friendly way. Also in the creation of textiles it harbors some advantages, since it can be used for the production of antimicrobial textiles plus paper or polymer materials may be produced with this versatile small organism. Arthrospira species among those useful herein include Arthrospira amethystine, Arthrospira ardissonei, Arthrospira argentina, Arthrospira balkrishnanii, Arthrospira baryana, Arthrospira braunii, Arthrospira breviarticulata, Arthrospira brevis, Arthrospira brevis, Arthrospira curta, Arthrospira desikacharyiensis, Arthrospira funiformis, Arthrospira fusiformis, Arthrospira ghannae, Arthrospira gigantean, Arthrospira gomontiana, Arthrospira indica, Arthrospira jenneri, Arthrospira joshii, Arthrospira khannae, Arthrospira laxa, Arthrospira laxissima, Arthrospira leopoliensis, Arthrospira major, Arthrospira margaritae, Arthrospira massartii, Arthrospira maxima, Arthrospira meneghiniana, Arthrospira miniata, Arthrospira neapolitana, Arthrospira nordstedtii, Arthrospira oceanica, Arthrospira okensis, Arthrospira pellucida, Arthrospira platensis, Arthrospira santannae, Arthrospira setchellii, Arthrospira skujae, Arthrospira spirulinoides, Arthrospira subsalsa, Arthrospira subtilissim, Arthrospira tenui, Arthrospira tenuissima, and Arthrospira versicolor.

Chlorella is a potential food source because it is high in protein and other essential nutrients; when dried, it is about 45% protein, 20% fat, 20% carbohydrate, 5% fiber, and 10% minerals and vitamins. When first harvested, Chlorella was suggested as an inexpensive protein supplement to the human diet. Advocates sometimes focus on other supposed health benefits of the algae, such as claims of weight control, improves the immune system, reduces blood lipids, antitumor action, health promoting effects on gastric ulcers, wounds, constipation, preventive action against atherosclerosis and hypercholesterolemia. Under certain growing conditions, Chlorella yields oils that are high in polyunsaturated fats—for example, Chlorella minutissima has yielded eicosapentaenoic acid, an omega-3 fatty acid (“EPA”) at 39.9% of total lipids. Many people believe Chlorella can serve as a potential source of food and energy because its photosynthetic efficiency can, in theory, reach 8% which exceeds that of other highly efficient crops such as sugar cane. Chlorella cells contain β-1,3-glucan, an active immunostimulator, which acts as a free-radical scavenger and as a reducer of blood lipids. A polysaccharide also found in Chlorella has been linked to antitumor effects and contains “CGF” (Chlorella growth factor), as well as other health-promoting substances. Chlorella species among those useful herein include Chlorella Acuminate, Chlorella Autotrophica, Chlorella Bacteroidea, Chlorella Botryoides, Chlorella Chlorelloides, Chlorella Colonialis, Chlorella Conglomerate, Chlorella Desiccate, Chlorella, Ellipsoidea, Chlorella Elongate, Chlorella Faginea, Chlorella Globose, Chlorella Glucotropha, Chlorella Heliozoae, Chlorella Infusionum, Chlorella Lewinii, Chlorella Marina, Chlorella Miniata, Chlorella Minima, Chlorella Minor, Chlorella Minuscula, Chlorella minutissima, Chlorella Nocturna, Chlorella Nordstedtii, Chlorella Oocystoides, Chlorella Ovalis, Chlorella Peruviana, Chlorella Photophila, Chlorella Pituita, Chlorella Pulchelloides, Chlorella Pyrenoidosa, Chlorella Regularis, Chlorella Rotunda, Chlorella Rugose, Chlorella Salina, Chlorella Singularis, Chlorella Sorokiniana, Chlorella Spaerckii, Chlorella Sphaerica, Chlorella Spp, Chlorella Stigmatophora, Chlorella Suboblonga, Chlorella Tertia, Chlorella Trebouxioides, Chlorella Ultrasquamata, Chlorella Umbelloidea, Chlorella Vannielii, Chlorella Variabilis, Chlorella Variegate, Chlorella Viridis, Chlorella Viscosa, Chlorella Volutis, Chlorella Vulgaris, and Chlorella Zofingiens is .

Crypthecodinium cohnii and Schizochytrium sp. are two microorganisms used for the commercial production of polyunsaturated fatty acids (PUFAs). Schizochytrium sp. is a heterotrophic microalga belonging to the order Thraustochytriales within the phylum Heterokonta, which can yield about 40% (w/w) of Docosahexaenoic acid (“DHA”) from its total fatty acid production. Crypthecodinium cohnii is a unique heterotrophic marine dinoflagellate in that DHA is almost exclusively the only PUFA present in its lipid and can be as high as 65% of the total fatty acids. Both have been shown to treat brain and heart disorders and to help brain development. Approximately 90% of all infant formulas contain DHA from these algae strains. In various embodiments, these organisms include Crypthecodinium cohnii, Crypthecodinium setense, Schizochytrium sp.

Dunaliella species belong to the phylum Chlorophyta, order Volvocales and family Polyblepharidaceae, and are unicellular, photosynthetic and motile biflagellate microalgae morphologically distinguished by the lack of a rigid cell wall. Many different species of Dunaliella can accumulate significant amounts of valuable fine chemicals such as carotenoids, glycerol, lipids, vitamins, minerals and proteins. Dunaliella is known for its capacity to produce high concentrations of carotenes, especially β-carotene plus contain valuable carotenoid pigments such as a-carotene, violaxanthin, neoxanthin, zeaxanthin and lutein. Dunaliella has positive influences on the energy metabolism of the skin, anticancer, antioxidant, antiviral. They also have a large potential for biotechnological processes such as expressing of foreign proteins and treatment of wastewater. Dunaliella species among those useful herein include Dunaliella acidophila, Dunaliella asymmetrica, Dunaliella baas, Dunaliella bardawil, Dunaliella bioculata, Dunaliella carpatica, Dunaliella cordata, Dunaliella euchlora, Dunaliella gracilis, Dunaliella granulate, Dunaliella jacobae, Dunaliella lateralis, Dunaliella maritima, Dunaliella media, Dunaliella minuta, Dunaliella minutissima, Dunaliella parva, Dunaliella peircei, Dunaliella polymorpha, Dunaliella primolecta, Dunaliella pseudosalina, Dunaliella quartolecta, Dunaliella ruineniana, Dunaliella salina, Dunaliella terricola, Dunaliella tertiolecta, Dunaliella turcomanica, and Dunaliella viridis.

Euglena may serve as a particularly valuable food source, containing essential vitamins, minerals, amino acids, and fatty acids. A major biological feature of euglena is the presence of a cell membrane instead of a cell wall found in most plants. A cell wall inhibits digestion since it requires cellulase, an enzyme not found in the human body to be decomposed. However, Euglena is only surrounded by a cell membrane which allows efficient digestion absorption. In addition, euglena can also be used as animal feeds instead of crops. This would contribute not only to change agricultural practices but also to enhance the quality and health status of livestock. Euglena algae can accumulate as much as 95% of the cell mass as beta-glucan known as paramylon. Because of their ability to enhance infection defense mechanisms and simultaneously downregulate inflammations, β-glucans are very promising as an alternative to the mainstream use of immunosuppressive drugs to treat inflammatory diseases. They may form part of a prophylaxis and/or therapeutic strategy following exposure to pathogen challenge, to reduce the need for antibiotics in both human and veterinary medicine, either as dietary supplement or pharmaceutical preparation. Euglena species among those useful herein include Euglena acauda, Euglena acaulis, Euglena acus, Euglena acusformis, Euglena acutata, Euglena acutissima, Euglena adhaerens, Euglena adunca, Euglena agilis, Euglena ahi, Euglena alata, Euglena allorgei, Euglena amblyophis, Euglena americana, Euglena amphipyrenica, Euglena anabaena, Euglena anaerobica, Euglena anguillula, Euglena angusta, Euglena angusta, Euglena antefossa, Euglena anura, Euglena aquaepurae, Euglena araci, Euglena arai, Euglena archaeoplastidiata, Euglena archaeoviridis, Euglena ascusformis, Euglena astasioides, Euglena aumuelleri, Euglena austalica, Euglena australica, Euglena bacillaria, Euglena bacilliformis, Euglena baltica, Euglena basistellata, Euglena bellovacensis, Euglena bichloris, Euglena bistellata, Euglena bivittata, Euglena boirsensis, Euglena bonettoi, Euglena brevicaudata, Euglena brevifilagellum, Euglena bucharica, Euglena caballeroi, Euglena calva, Euglena cantabrica, Euglena cantabrica, Euglena carteriae, Euglena caudata, Euglena centralis, Euglena centrorubra, Euglena chadefaudii, Euglena chaetophorina, Euglena chamberlinii, Euglena charkowiensis, Euglena chlamydophora, Euglena chlorodictyon, Euglena chlorophoenicea, Euglena choretes, Euglena cingula, Euglena circularis, Euglena clara, Euglena clavata, Euglena communis, Euglena compressa, Euglena concavus, Euglena confusa, Euglena convoluta, Euglena copula, Euglena crescentia, Euglena cuneata, Euglena curvata, Euglena cuvata, Euglena cyclopicola, Euglena cylindrica, Euglena demulcens, Euglena deses, Euglena detonii, Euglena dicentra, Euglena dikaryon, Euglena discolor, Euglena distincat, Euglena downiae, Euglena durbanica, Euglena ecaudata, Euglena ehrenbergii, Euglena elastica, Euglena elenkinii, Euglena elongata, Euglena estonica, Euglena ettlii, Euglena eutreptia, Euglena excavata, Euglena exilis, Euglena fenestrata, Euglena flagellata, Euglena flava, Euglena foliacea, Euglena fornicata, Euglena fracta, Euglena fronsundulata, Euglena fundoversata, Euglena fusca, Euglena fusiformis, Euglena gasterosteus, Euglena gaumei, Euglena geniculata, Euglena geniculata, Euglena gentilis, Euglena gibbosa, Euglena gigas, Euglena globosa, Euglena gojdicsas, Euglena gracilis, Euglena granulata, Euglena grisolii, Euglena guentheri, Euglena guttula, Euglena gymnodinioides, Euglena haematodes, Euglena heimii, Euglena helicoidea, Euglena heliorubescens, Euglena hemichromata, Euglena hiemalis, Euglena hiemii, Euglena hirudo, Euglena hispidula, Euglena hyalina, Euglena iara, Euglena ignobilis, Euglena incisa, Euglena incurva, Euglena inflate, Euglena inflexa, Euglena intermedia, Euglena interrupta, Euglena intervolans, Euglena iraci, Euglena irai, Euglena jacira, Euglena jandira, Euglena jirovecii, Euglena juraci, Euglena kemenesii, Euglena klebsii, Euglena korshikovii, Euglena laciniata, Euglena laevis, Euglena lata, Euglena lepocincloides, Euglena leucops, Euglena limnophila, Euglena limosa, Euglena longa, Euglena longicauda, Euglena longicaudata, Euglena longissima, Euglena longuscula, Euglena lucens, Euglena lutaria, Euglena magnifica, Euglena maharastrensis, Euglena mainxi, Euglena mainxii, Euglena mangenotti, Euglena mangini, Euglena matvienkoi, Euglena megalithos, Euglena mesnilii, Euglena messula, Euglena metabolica, Euglena middelhoekii, Euglena minima, Euglena minuta, Euglena montanensis, Euglena mucifera, Euglena mucosa, Euglena mucronata, Euglena multiformis, Euglena mutabilis, Euglena myxocylindracea, Euglena nana, Euglena navicula, Euglena neglecta, Euglena neustonica, Euglena oblonga, Euglena obscura, Euglena obtusa, Euglena obtusa-caudata, Euglena olivacea, Euglena orientalis, Euglena ornate, Euglena orthia, Euglena ostendensis, Euglena ovum, Euglena oxyuris, Euglena pailasensis, Euglena palmeri, Euglena paludosa, Euglena paradoxa, Euglena parasitica, Euglena pascheri, Euglena pavlovskoensis, Euglena pedunculata, Euglena penardii, Euglena phacoides, Euglena physeter, Euglena pigmaea, Euglena pisciformis, Euglena platydesma, Euglena pleuronectes, Euglena polymorpha, Euglena pringsheimii, Euglena pringsheimii, Euglena prowsei, Euglena proxima, Euglena pseudocentralis, Euglena pseudochadefaudii, Euglena pseudoehrenbergii, Euglena pseudomermis, Euglena pseudospirogyra, Euglena pseudospiroides, Euglena pseudostellata, Euglena pseudotuba, Euglena pseudoviridis, Euglena pseudoxyuris, Euglena pumila, Euglena purpurea, Euglena pusilla, Euglena pyriformis, Euglena pyrum, Euglena quartana, Euglena radians, Euglena radiate, Euglena ranunculuformis, Euglena refringens, Euglena repulsans, Euglena reticulate, Euglena retronata, Euglena rhynchophora, Euglena rivulariarum, Euglena roberti-lamii, Euglena rostrate, Euglena rostrifera, Euglena rubida, Euglena rubra, Euglena rustica, Euglena ruttneri, Euglena sabulorum, Euglena sacculiformis, Euglena salina, Euglena sanguinea, Euglena satelles, Euglena scherffelii, Euglena schmitzii, Euglena sciotensis, Euglena seppiana, Euglena sessilis, Euglena sieminskiana, Euglena sima, Euglena simulacra, Euglena slavjanskiensis, Euglena smulkowskiana, Euglena sociabilis, Euglena spadix, Euglena spathirhyncha, Euglena sphagnicola, Euglena spinifera, Euglena spirogyra, Euglena spiroides, Euglena splendens, Euglena srinagari, Euglena stellate, Euglena striato-punctata, Euglena subacutissima, Euglena subangusta, Euglena subehrenbergii, Euglena subthinophila, Euglena sulcate, Euglena sulcifera, Euglena swirenkoi, Euglena synchlora, Euglena tatrica, Euglena tentans, Euglena terricola, Euglena texta, Euglena thienemannii, Euglena thinophila, Euglena tibetica, Euglena tiszae, Euglena tornata, Euglena torta, Euglena triptereris, Euglena tripteris, Euglena triquetra, Euglena tristella, Euglena trisulcata, Euglena truncate, Euglena truncatula, Euglena tuba, Euglena tuberculate, Euglena undulata, Euglena univittata, Euglena utriculus, Euglena vagans, Euglena vaginicola, Euglena van-goori, Euglena variabilis, Euglena velata, Euglena velveta, Euglena vermicularis, Euglena vermiformis, Euglena vesterbottnica, Euglena vilmae, Euglena viridis, Euglena vittata, Euglena vivida, Euglena walneae, Euglena wangii, Euglena zocchioi, and Euglena zonalis.

Haematococcus are among the commercially important microalgae. For example, Haematococcus pluvialis is the richest natural source of astaxanthin which is considered to be a “super anti-oxidant.” Natural astaxanthin produced by H. pluvialis has significantly greater antioxidant capacity than synthetic astaxanthin. Astaxanthin has important applications in the nutraceuticals, cosmetics, food, and aquaculture industries. It is now evident that, astaxanthin can significantly reduce free radicals and oxidative stress and help the human body maintain a healthy state. Animal studies have shown that astaxanthin can protect skin from UV radiation effects, protect against chemically induced cancers, and enhance the immune system. With extraordinary potency and increase in demand, astaxanthin is one of the high-value microalgal products of the future. Astaxanthin (3,3′-dihydroxy-β-carotene-4,4′-dione) is a bright red secondary carotenoid from the same family as lycopene, lutein, and β-carotene, synthesized de novo by some microalgae, plants, yeast, bacteria, and present in many of our favored seafood including salmon, trout, red sea bream, shrimp, lobster and fish eggs. Haematococcus among those useful herein include Haematococcus allmanii, Haematococcus buetschlii, Haematococcus capensis, Haematococcus carocellus, Haematococcus cordae, Haematococcus dalmaticus, Haematococcus droebakensis, Haematococcus frustulosus, Haematococcus grevillea, Haematococcus hookeriana, Haematococcus insignis, Haematococcus kermesinus, Haematococcus lacustris, Haematococcus longistigma, Haematococcus murorum, Haematococcus pluvialis, Haematococcus rubens, Haematococcus rubicundus, Haematococcus salinus, Haematococcus sanguineus, Haematococcus thermalis, and Haematococcus zimbabwiensis.

Isochrysis is capable of building stores of fats and oils. Isochrysis is high in polyunsaturated fatty acids such as DHA, stearidonic acid and alpha-linolenic acid. The Omega 3 composition of I. galbana is often over 22% total fatty acid content (the so-called Tahitian strain of Isochrysis is said to be especially rich in DHA). It is frequently used to enrich live zooplanktonic feeds (rotifers, copepods, brine shrimp, etc.). It has been used effectively to feed very demanding phytoplanktivorous corals such as Dendronephthya. The genus Isochrysis also contains Tisochrysis lutea. T. lutea is one of the most widely used species in aquaculture to feed oyster and shrimp larvae. It has an interesting composition for this application because of its high content of polyunsaturated fatty acids such as DHA, stearidonic acid and alpha-linolenic acid. T. lutea contain betain lipids and phospholipids. Isochysis among those useful herein include Isochrysis galbana, Isochrysis litoralis, Isochrysis maritima, Isochrysis nuda, Isochrysis santou, and Isochrysis zhanjiangensis.

Nannochloropsis were first identified in the early 1980′s and since then have been extensively researched in Europe for their industrial applications beneficial for aquaculture, biofuel, cosmetics as well as for nutritional supplementation purposes. Nannochloropsis is considered a promising algae for industrial applications because of its ability to accumulate high levels of polyunsaturated fatty acids. Moreover, it shows promising features that can allow genetic manipulation aimed at the genetic improvement of the current oleaginous strains. In addition they are able to build up high concentrations of a range of pigments such as astaxanthin, zeaxanthin and canthaxanthin. As a highly nutritious micronutrient-rich substance, it is an excellent way to provide an immediate influx of omega-3 fatty acids, vitamins, minerals, amino acids, superoxide dismutase and many carotenoid antioxidants in an easily absorbed and assimilated liquid. At the moment it is mainly used as an energy-rich food source for fish larvae and rotifers. Nannochloropsis among those useful herein include Nannochloropsis australis, Nannochloropsis gaditana, Nannochloropsis granulate, Nannochloropsis limnetica, Nannochloropsis oceanica, Nannochloropsis oculata, Nannochloropsis salina, and Nannochloropsis sp.

The microalgae of the Odontella genus are unicellular algae of large size, the length of which may attain 35 to 50 microns and are very abundant throughout the oceans. Odontella aurita is a marine diatom rich in EPA as well as in other bioactive molecules, such as pigments, and has use in animal food, notably for feeding larvae of fish and crustaceans. 0. aurita can be a natural source of fucoxanthin for human health and nutrition and is being commercially used for human consumption. Odontella among those useful herein include Odontella affinis, Odontella amphicephala, Odontella angulate, Odontella antediluviana, Odontella atlantica, Odontella aurita, Odontella barbadensis, Odontella biddulphioides, Odontella birostrum, Odontella brittoniana, Odontella calamus, Odontella chinensis, Odontella cookiana, Odontella cornuta, Odontella cristata, Odontella decorate, Odontella desmidium, Odontella discigera, Odontella dissipata, Odontella dubia, Odontella edwardsii, Odontella elegans, Odontella expedita, Odontella favus, Odontella febigeri, Odontella fenestrate, Odontella filiformis, Odontella fimbriata, Odontella gallapagensis, Odontella granulate, Odontella hastate, Odontella heibergii, Odontella laevis, Odontella litigiosa, Odontella longicruris, Odontella macdonaldii, Odontella mammosa, Odontella manca, Odontella minutissima, Odontella mobiliensis, Odontella neogradensis, Odontella oamaruensis, Odontella obtuse, Odontella parva, Odontella pedalis, Odontella plana, Odontella polycanthos, Odontella polymera, Odontella polymorpha, Odontella primordialis, Odontella pygmea, Odontella regia, Odontella reticulate, Odontella retiformis, Odontella rhomboids, Odontella rhombus, Odontella roperiana, Odontella rostrate, Odontella separanda, Odontella septentrionala, Odontella septentrionalis, Odontella spinose, Odontella spinulosa, Odontella striata, Odontella subaequa, Odontella sublevis, Odontella tentaculifera, Odontella tetraodon, Odontella tubulosa, Odontella turgida, Odontella unidentata, and Odontella weissflogii.

Phaeodactylum tricornutum is a diatom that has emerged in the biotechnology field for various applications, such as a biofuel precursor and recombinant protein expression host due to its biosynthetic capacity and high growth rates. Based on scientific evidence, some species of algae possess bioactive molecules that can be used to fight against postoperative infectious drug-resistant bacteria and Phaeodactylum has been reported to produce antimicrobial compounds. Phaeodactylum useful herein include Phaeodactylum reichelti, and Phaeodactylum tricornutum.

Picochlorum, a newly established genus, including species previously included in the genus Nannochloris, is a member of division Chlorophyta and class Trebouxiophyceae. Most of these organisms are small coccoid (non-motile), asexual, unicellular, green algae, and can withstand hypersaline conditions. Some species of Picochlorum are well studied and contain chlorophyll a and b as the major pigments in addition to the carotenoids such as lutein, β-carotene, violaxanthin, neoxanthin and vaucheriaxanthin. Picochlorum oklahomensis is reported to have a higher growth rate of 0.5 μ/day, 0.7 divisions per day, and a shorter generation time of 1.4 days. Biochemical analysis of Picochlorum oklahomensis showed that it contained 20% lipids and 35% protein of the total dry weight, and fatty acid methyl ester (“FAME”) analyses showed 27% saturated fatty acids, 22% monounsaturated fatty acids, and 46% polyunsaturated fatty acids of the total lipid. Because of high protein and oil content, the alga is suitable for the food and feed industries. The oxidative stability from P. oklahomensis may be low due to its high unsaturated fatty acid content, high PUFA, specifically, linoleic and linolenic acid contents, which enhances the high nutritional value of algal biomass produced. Picochlorum among those useful herein include Picochlorum atomus, Picochlorum eukaryotum, Picochlorum maculatum, Picochlorum oculatum, and Picochlorum oklahomense.

Porphyridium is a potential source for several products like fatty acids, lipids, cell-wall polysaccharides and pigments. The polysaccharides of this species are sulphated and their structure gives rise to some unique properties that could lead to a broad range of industrial and pharmaceutical applications. Additionally, P. cruentum biomass contains carbohydrates of up to 57% have been reported. Thus, the combined amount of carbohydrates in biomass and exopolysaccharides of this microalga could potentially provide the source for bio-fuel and pharmaceutical compounds. Porphyridium cruentum contains relatively rare polyunsaturated fatty acids such as arachidonic acid and EPA, both important in human nutrition. Porphyridium species among those useful herein include Porphyridium aerugineum, Porphyridium cruentum, Porphyridium griseum, Porphyridium marinum, Porphyridium purpureum, Porphyridium schinzii, Porphyridium sordidum, Porphyridium violaceum, Porphyridium wittrockii.

Scenedesmus grows abundantly plus offers important applications for animal and human consumption. For instance, this strain produces large quantities of carotenoids particularly lutein and beta-carotene. Scenedesmus is more commonly known as a source of food for herbivorous zooplankton and in biofuel production because of its high lipid content. It has been used in many biotechnological applications due to its high nutritional content and bioactivities. They have been exploited for their active metabolites that have been applied in various industries including pharmaceutical, food, cosmetics, energy, aquaculture, medicine and others. Many species of this genus are being used worldwide for various purposes due to their ability to adapt to harsh environmental conditions, ability to grow rapidly and ease of cultivation and handling. Scenedesmus has exhibited the potential of being a source of high-value compounds with antibacterial properties. These antibacterial activities have a wide range of applications in various industries that have not been broadly explored and fully exploited. Scenedesmus among those useful herein include Scenedesmus abundans, Scenedesmus aciculatus, Scenedesmus aculeato-granulatus, Scenedesmus aculeolatus, Scenedesmus aculeotatus, Scenedesmus acuminatus, Scenedesmus acunae, Scenedesmus acutiformis, Scenedesmus acutus, Scenedesmus alatus, Scenedesmus aldavei, Scenedesmus alternans, Scenedesmus ambuehlii, Scenedesmus anhuiensis, Scenedesmus ankistrodesmoides, Scenedesmus annandalei, Scenedesmus anomalus, Scenedesmus antennatus, Scenedesmus antillarum, Scenedesmus apicaudatus, Scenedesmus apiculatus, Scenedesmus arcuatus, Scenedesmus aristatus, Scenedesmus armatus, Scenedesmus arthrodesmiformis, Scenedesmus arvernensis, Scenedesmus asymmetricus, Scenedesmus bacillaris, Scenedesmus baculiformis, Scenedesmus bajacalifornicus, S cenedesmus balatonicus, Scenedesmus basiliensis, Scenedesmus bellospinosus, Scenedesmus berczikii, Scenedesmus bernardii, Scenedesmus bicaudatus, Scenedesmus bicaudatus, Scenedesmus bicellularis, Scenedesmus bidentatus, Scenedesmus bijugatus, Scenedesmus bijugus, Scenedesmus bourrellyi, Scenedesmus brasiliensis, Scenedesmus breviaculeatus, Scenedesmus brevispina, Scenedesmus budhaensis, Scenedesmus capitato-aculeatus, Scenedesmus carabus, Scenedesmus caribeanus, Scenedesmus carinatus, Scenedesmus caudate, Scenedesmus caudato-aculeolatus, Scenedesmus caudatus, Scenedesmus chlorelloides, Scenedesmus circumfusus, Scenedesmus clathratus, Scenedesmus coalitus, Scenedesmus coelastroides, Scenedesmus columnatus, Scenedesmus communis, Scenedesmus corallines, Scenedesmus co s tato-granulatu s, Scenedesmus co s tatu s, Scenedesmus costulatus, Scenedesmus courbetensis, Scenedesmus crassidentatus, Scenedesmus crassus, Scenedesmus cumbricus, Scenedesmus cuneatus, Scenedesmus curvatocornis, Scenedesmus curvatus, Scenedesmus dactylococcoides, Scenedesmus danubialis, Scenedesmus decorus, Scenedesmus denticulatus, Scenedesmus deserticola, Scenedesmus diagonalis, Scenedesmus dileticus, Scenedesmus dimorphus, Scenedesmus disciformis, Scenedesmus dispar, Scenedesmu s dissociates, Scenedesmus distentus, Scenedesmus dividuus, Scenedesmus echinulatu s, Scenedesmus ecornis, Scenedesmus elegans, Scenedesmus ellipsoideus, Scenedesmus ellipticus, Scenedesmus eupectinatus, Scenedesmus falcatus, Scenedesmus fenestratus, Scenedesmus flavescens, Scenedesmus flexuosus, Scenedesmus fuegiensis, Scenedesmus furcato-spinulosus, Scenedesmus furcosus, Scenedesmus fuscus, Scenedesmus fusiformis, Scenedesmu s ginzenbergii, Scenedesmus gracilis, Scenedesmus gracils, Scenedesmus graevenitzii, Scenedesmus grahneisii, Scenedesmus granulatus, Scenedesmu s guarrerae, Scenedesmu s gujaratensis, Scenedesmus gutwinskii, Scenedesmus hanleyi, Scenedesmus heimii, Scenedesmus helveticus, Scenedesmus heteracanthus, Scenedesmus hindakii, Scenedesmus hirsutus, Scenedesmus hirtutus, Scenedesmus hortobagyi, Scenedesmus houlensis, Scenedesmus huangshanensis, Scenedesmus hunanensis, Scenedesmus hystrix, Scenedesmus incras satulus, Scenedesmus indianensis, Scenedesmus indicus, Scenedesmus inermis, Scenedesmus insignis, Scenedesmus intermedius, Scenedesmus irregularis, Scenedesmus javanensis, Scenedesmus jovis, Scenedesmus kantonensis, Scenedesmus kerguelensis, Scenedesmus kis sii, Scenedesmus kolhapurensis, Scenedesmus komarekii, Scenedesmus lacustris, Scenedesmus lefevrei, Scenedesmus linearis, Scenedesmus littoralis, Scenedesmus longicauda, Scenedesmus longicornis, Scenedesmus longispina, Scenedesmus longus, Scenedesmus luna, Scenedesmus lunatus, Scenedesmus maculosus, Scenedesmus magnogranulatus, Scenedesmus magnus, Scenedesmus maharastrensis, Scenedesmus margalefii, Scenedesmus maximus, Scenedesmus micro spina, Scenedesmus minutus, Scenedesmus mirificus, Scenedesmus mirus, Scenedesmus monomorphus, Scenedesmus morzinensis, Scenedesmus multicauda, Scenedesmus multiformis, Scenedesmus multispina, Scenedesmus multistriatus, Scenedesmus muzzanensis, Scenedesmus naegelii, Scenedesmus nanu s, Scenedesmus notatus, Scenedesmus nygaardii, Scenedesmus oahuensis, Scenedesmus obliquus, Scenedesmus obtusiusculus, Scenedesmus obtusus, Scenedesmus octocauda, Scenedesmus oocystiformis, Scenedesmus opoliensis, Scenedesmus ornatus, Scenedesmus ovalternus, Scenedesmus ovalternus, Scenedesmus pannonicus, Scenedesmus papillatus, Scenedesmus papillosum, Scenedesmus papillosus, Scenedesmus parisiensis, Scenedesmus parvus, Scenedesmus pecsensis, Scenedesmus pectinatus, Scenedesmus pelvis, Scenedesmus perforates, Scenedesmus philiposei, Scenedesmus planctonicus, Scenedesmus platydiscus, Scenedesmus pleiomorphus, Scenedesmus polessicus, Scenedesmus polycostatus, Scenedesmus polydenticulatus, Scenedesmus polyglobulus, Scenedesmus praetervisus, Scenedesmus prismaticus, Scenedesmus priyankaii, Scenedesmus producto-capitatus, Scenedesmus protuberans, Scenedesmus pseudasymetricus, Scenedesmus pseudo-alatus, Scenedesmus pseudoarmatus, Scenedesmus pseudobernardii, Scenedesmus pseudodenticulatus, Scenedesmus pseudogranulatus, Scenedesmus pseudohystrix, Scenedesmus pseudolunatus, Scenedesmus pseudoquadrcauda, Scenedesmus pulloideus, Scenedesmus pupukensis, Scenedesmus pyrus, Scenedesmus quadrialatus, S cenedesmus quadric auda, Scenedesmus quadrispina, Scenedesmus raciborskii, Scenedesmus ralfsii, Scenedesmus reginae, Scenedesmus regularis, Scenedesmus reniformis, Scenedesmus rostrato- spinosus, Scenedesmus rotundus, Scenedesmus rubescens, Scenedesmus scenedesmoides, Scenedesmus schnepfii, Scenedesmus schroeteri, Scenedesmus scutatus, Scenedesmus securiformis, Scenedesmus semicristatus, Scenedesmus semipulcher, Scenedesmus sempervirens, Scenedesmus senilis, Scenedesmus serrato-perforatus, Scenedesmus serratus, Scenedesmus setiferus, Scenedesmus sihensis, Scenedesmus similagineus, Scenedesmus smithii, Scenedesmus soli, Scenedesmus sooi, Scenedesmus speciosus, Scenedesmus spicatus, Scenedesmus spinoso-aculeolatus, Scenedesmus spinosus, Scenedesmus spinulatus, Scenedesmus striatus, Scenedesmus subspicatus, Scenedesmus tenuispina, Scenedesmus terrestris, Scenedesmus tetradesmiformis, Scenedesmus trainorii, Scenedesmus transilvanicus, Scenedesmus tricostatus, Scenedesmus tropicus, Scenedesmus tschudyi, Scenedesmus unicellularis, Scenedesmus ushuaiensis, Scenedesmus vacuolatus, Scenedesmus varians, Scenedesmus velitaris, Scenedesmus verrucoso-costatus, Scenedesmus verrucosus, Scenedesmus vesiculosus, Scenedesmus westii, Scenedesmus wisconsinensis, Scenedesmus wuhanensis, Scenedesmus wuhuensis, Scenedesmus wuxiensis, and Scenedesmus yiduensis.

Spirulina are rich in nutrients, some of which aren't found in the average daily vitamin. According to the FDA, the health benefits of Spirulina include significant amounts of calcium, niacin, potassium, magnesium, B vitamins and iron. Spirulina is a great source of B complex vitamins, beta-carotene, vitamin E, manganese, zinc, copper, iron, selenium and antioxidants. Spirulina also contain essential amino acids (compounds that are the building blocks of proteins). Spirulina are one of the richest algal sources of γ-linolenic acid (“GLA”) and GLA is an essential polyunsaturated fatty acid and a potent nutraceutical. Various studies have asserted its pharmaceutical value, especially in lowering the low-density lipoprotein in hypercholesterolemic patients and alleviation of symptoms in premenstrual syndrome and atopic eczema. Spirulina are also an excellent source of phycobiliproteins. These compounds are being studied due to their high free-radical scavenging capacity, which could make them a potential antitumor and anticancer drug. Spirulina may have immunoenhancing prevention, assist treatment of heart diseases, obesity, manic depression, antitumor effect, antioxidant activity, nutritional supplements to inhibit replication and infectivity of virus including HIV, CMV, HSV, and influenza A. Due to the easy bioavailability of nutrients, including minerals, Spirulina may be a good choice for women during pregnancy and lactation and is also beneficial for malnourished children. The World Health Organization (WHO) has called Spirulina as one of the greatest superfoods on earth and NASA considers it as an excellent compact food for space travel, as a small amount can provide a wide range of nutrients. Spirulina has been incorporated in noodles, cookies, nutritional bars, and other functional food products and also supports digestive functions by helping to maintain bacteria in the gut. Spirulina among those useful herein include Spirulina abbreviate, Spirulina adriatica, Spirulina aeruginea, Spirulina agilis, Spirulina agilissima, Spirulina albida, Spirulina allansonii, Spirulina amethystine, Spirulina anjalensis, Spirulina ardissonei, Spirulina ardissoni, Spirulina argentina, Spirulina attenuata, Spirulina baltica, Spirulina bayannurensis, Spirulina breviarticulata, Spirulina cabrerae, Spirulina caldaria, Spirulina cavanillesiana, Spirulina compacta, Spirulina condensate, Spirulina conica, Spirulina corakiana, Spirulina curta, Spirulina duplex, Spirulina erdosensis, Spirulina flavovirens, Spirulina funiformis, Spirulina gessneri, Spirulina gigantean, Spirulina gomontiana, Spirulina gomontii, Spirulina gordiana, Spirulina gracilis, Spirulina gracillima, Spirulina innatans, Spirulina jenneri, Spirulina labyrinthiformis, Spirulina laxa, Spirulina laxissima, Spirulina legitima, Spirulina magnifica, Spirulina major, Spirulina margaritae, Spirulina mariae, Spirulina massartii, Spirulina maxima, Spirulina mediterranea, Spirulina meneghiniana, Spirulina miniata, Spirulina minima, Spirulina mukdensis, Spirulina neumannii, Spirulina nodosa, Spirulina nordstedtii, Spirulina oceanica, Spirulina okensis, Spirulina oscillarioides, Spirulina platensis, Spirulina princeps, Spirulina pseudotenuissima, Spirulina pseudovacuolata, Spirulina regis, Spirulina rhaphidioides, Spirulina robusta, Spirulina rosea, Spirulina schroederi, Spirulina sigmoidea, Spirulina socialis, Spirulina spirulinoides, Spirulina stagnicola, Spirulina subsalsa, Spirulina subtilissima, Spirulina supersalsa, Spirulina tenerrima, Spirulina tenuior, Spirulina tenuis, Spirulina tenuissima, Spirulina thermalis, Spirulina turfosa, Spirulina undulans, Spirulina versicolor, Spirulina weissii, and Spirulina spp.

Tetraselmis are found in both marine and freshwater ecosystems across the globe; thus, they live in diverse water-environments if enough nutrients and light is available for net photosynthetic activity. The microalga Tetraselmis is a potential source of economically valuable EPA and DHA. Tetraselmis' are used as food in aquaculture, and for biotechnology uses which have proven to be beneficial for both research and industry. Tetraselmis has a very high lipid level plus their amino acids stimulate feeding in marine organisms. They have been studied for understanding plankton growth rates. Tetraselmis species are a promising source for biofuel use due to their fast growth rate, high lipid content, cheaper photosynthetic mechanisms, less need for agricultural land, useful by-products and for being environmentally friendly. Tetraselmis among those useful herein include Tetraselmis alacris, Tetraselmis apiculate, Tetraselmis arnoldii, Tetraselmis ascus, Tetraselmis astigmatica, Tetraselmis bichlora, Tetraselmis bilobata, Tetraselmis bolosiana, Tetraselmis chui, Tetraselmis contracta, Tetraselmis convolutae, Tetraselmis cordiformis, Tetraselmis desikacharyi, Tetraselmis elliptica, Tetraselmis fontiana, Tetraselmis gracilis, Tetraselmis hazenii, Tetraselmis helgolandica, Tetraselmis impellucida, Tetraselmis incisa, Tetraselmis inconspicua, Tetraselmis indica, Tetraselmis levis, Tetraselmis limnetis, Tetraselmis maculate, Tetraselmis marina, Tetraselmis mediterranea, Tetraselmis micropapillata, Tetraselmis rubens, Tetraselmis striata, Tetraselmis subcordiformis, Tetraselmis suecica, Tetraselmis tetrathele, Tetraselmis verrucosa, Tetraselmis viridis, and Tetraselmis wettsteinii.

In various embodiments, the compositions and methods of the present technology employ phototropic production of Haematococcus, Nannochloropsis, Picochlorum, Chlorella, Spirulina, and Dunaliella. In various embodiments, compositions and methods employ heterotrophic or mixotrophic production of Euglena. In particular, in various embodiments, the compositions and methods of the present technology employ algae selected from the group consisting of Haematococcus species (e.g. H. pluvialis), Chlorella species (e.g. C. vulgaris, C. zofringiensis), Chaetoceros sp., Dunaliella sauna, Phaeodactylum tricornutum, Porphyridium cruentum, Rhodella species, Skeletonema species, Scenedesmus species, and spirulina (e.g. Arthrospira maxima and Arthrospira platensis).

In various embodiments, beads comprise from about 20 up to about 90 percent algae. Exemplary beads contain a majority of algae (above 50% by weight of the bead) such as at least 50%, at least 55%, or at least 60% by weight. Beads are further characterized by not more than 85%, not more than 80%, not more than 75%, or not more than 70% by weight of the microalgae cells. In a non-limiting example, the beads contain about 60% to about 75% by weight of microalgae cells.

### Organic Polymer

The beads of the present technology also include a matrix comprising organic polymer. In various embodiments, the polymer is cross-linked in the presence of other components, e.g., algae, so as to produce a bead seed entraining or otherwise comprising such components. For example, the organic polymer undergoes a cross-linking reaction, for example with divalent cations, to provide a crosslinked organic matrix in the beads. The polymer before crosslinking is soluble in water and has a plurality of hydroxyl groups that react with the cations. Cros slinking the organic polymer in this way provides a reservoir containing microbe cells along with char and other components.

Examples of organic polymer include soluble polysaccharides such as, without limitation, alginate (especially sodium alginate), galactomannans, carrageenan, agar, agarose, carboxymethyl cellulose, gellan gum, gum tragacanth, dextran, methylcellulose, hydroxypropylmethyl cellulose, and guar gum. Galactomannans are polysaccharides consisting of a mannose backbone with galactose side groups. Examples include fenugreek gum, guar gum, tara gum, locust bean gum, and cassia gum.

Another class of organic polymers is the class of polyvinyl alcohol (“PVOH”). It can be crosslinked with di-cations or boric acid.

Crosslinking of the hydroxyl containing organic polymer may be provided by divalent cations in solution. The cations in one embodiment are those of Group II of the periodic table, known as the alkaline earth metals. Alkaline earth metal dications include Be⁺², Ca⁺², Mg⁺², Sr⁺², and Ba⁺². Of these, the most commonly used is Ca⁺². Calcium chloride is a good commercially-available source of divalent Ca⁺² cations.

In various embodiments, the crosslinking is achieved using cations corresponding to the nutrients required to grow a strain of algae before it is incorporated into the algal beads of the current teachings, such as described further below. These nutrients include soluble salts of cations such as those selected from Mg⁺², Ca⁺², Mn⁺², Zn⁺², Cu⁺², and Co⁺².

In various embodiments, beads comprise at least 1% by weight up to about 50% by weight of the crosslinked polymer. In alternative embodiments, the beads contain at least 2% or at least 3% polymer, which in the bead is present in a crosslinked, or gelled, form. In various embodiments, the beads contain no more than 40%, no more than 30%, or no more than 20% by weight polymer in the form of its crosslinked form. In a non-limiting example, the beads contain from about 3 to about 15% by weight of polymer.

### Water Insoluble Carbon Source

The beads comprise a water-insoluble carbon source, such as biochar or other char, comprising solid material that remains after light gases have been driven out or released during the initial stage of combustion (or pyrolysis) of a carbonaceous material, particularly under reduced oxygen levels or in the absence of oxygen. The process by which the char is produced is generally referred to as pyrolysis, carbonization, charring, devolatilization, and similar terms. The char product of the pyrolysis reaction is enriched in elemental carbon compared to the carbon content of the carbonaceous starting material. In various embodiments, the char is 80% or more carbon, or 90% or more by weight carbon. In addition to a major amount of carbon, the char normally also contains smaller amounts of ash derived from non-volatile components of the carbonaceous starting material.

Char may include materials such as charcoal, carbon black, coal black, char, and biochar. In various embodiments, the beads comprise biochar, which is made by pyrolysis of a biomass. Biomass sources include algae, rice hulls, corn stover, waste wood, nut shells, vineyard waste, manure, crop residues, grasses such as switchgrass, and agricultural waste. In various embodiments, biochar is derived from microalgae cells or rice hulls. In addition to charcoal powder, biochar normally contains inorganic minerals present in the biomass and not volatilized during the pyrolysis process. Biochar may be obtained from any of a variety of commercial sources, such as Charfecta Biochar UHP (Ultra High Porosity).

Char takes on a variety of structures and configurations depending on the nature of the carbon in the starting material and the conditions of pyrolysis used to make the char. In some embodiments, it is preferred to use char in the form of fine particles, such as ones characterized commercially as “ultrafine mesh.” Particular particle sizes and size distributions can be obtained by adjusting pyrolysis conditions in light of the nature of the carbonaceous material used as starting material for the pyrolysis reaction, and by grinding to produce a target size. Examples are char containing at least some particles that are 10 μm or less, 5 μm or less, 3 μm or less, 2 μm or less, or 1 μm or less, or in the sub-micron range. In an embodiment, at least some of the char particles are no bigger than the microalgae used in the beads.

Without limiting the scope, mechanism, function or utility of present technology, it is believed that, in various embodiments, char serves essentially as a carbon source for microalgae cultivation, in addition to its effect on the physical properties of beads containing the char. As discussed below, char is provided as a suspension in a microalgae culture before the beads are formed by crosslinking the organic matrix provided by the soluble polymer. In an aspect, the density of beads depends on the amount of char in the char suspension.

In various embodiments, beads comprise a minor amount of char, such as from about 0.5% up to about 20% by weight or up to about 10% by weight. In a non-limiting example, the beads of the present teachings contain from about 1 to about 6% by weight of insoluble carbon (e.g., biochar).

### Clay

Clay is an optional component of the beads. Examples include aluminosilicate clays, such as kaolin (which is a food grade material). Kaolin is commercially available, such as from CB Minerals, Mamaroneck, N.Y. Clay is believed to function as a pH modifier, and to assist with storing and releasing carbon, for example in the form of carbon dioxide. In an embodiment, particle size of the clay or aluminosilicate material is selected from those favored for the char particles described above.

Clay has the unique property of adsorption and absorption. Without limiting the scope, mechanism, function or utility of present technology, in various embodiments, clay can hold carbon dioxide (“CO23”) in its plate-like structures, such as in embodiments where CO2 is bubbled or otherwise introduced to beads during formation so as to produce a mixture bead having a diversity of densities, as discussed below. The CO2 is held and released as the pH of the water changes. To achieve a high concentration of CO2 in solution would require colder temperature and/or higher pressure. But colder temperature and high pressure cannot be used in algae production as it will kill the algae so entrapping the CO2 directly into clay is accomplished under conditions closer to ambient where the microalgae are not harmed. In some embodiments, entrapment of the CO2 in clay may provide sequestered carbon to feed the microalgae.

In various embodiments, beads contain a minor amount of clay, such as from about 0.5% up to about 20% by weight or up to about 10% by weight. In a non-limiting example, the beads of the present teachings contain from about 1 to about 6% by weight of clay.

### Nutrients

The beads optionally comprise microalgae nutrients. In general, photosynthetic microalgae grow in cellular culture if provided with carbon dioxide, photons of light, and various trace nutrients. Heterotrophic microalgae grow in cellular culture if provided with a carbon source and various trace minerals, even when there is no light. Mixotrophic microalgae grow in cellular culture if provided with either photosynthetic or heterotrophic conditions. The trace nutrients are part of the microalgae culture incorporated into beads as the organic polymer is crosslinked in situ.

Nutrients may include materials selected from the group consisting of trace metals, chelators, vitamins, soil extracts and mixtures thereof. For example, in various embodiments, beads optionally contain cations and anions corresponding to the nutrients required to grow a strain of algae before it is incorporated into the algal beads of the current teachings. These nutrients include soluble salts of cations such as those selected from Na⁺, K⁺, Mg⁺², Ca⁺², Fe⁺³, Mn⁺², Zn⁺², Cu⁺², and Co⁺², as well as soluble salts of one or more anions including those selected from nitrates, phosphates, hydrogen phosphates, dihydrogen phosphates, sulfates, chlorides, EDTA, carbonates, bicarbonates, and molybdates. In various embodiments, beads include divalent ions selected from those of Group II of the periodic table (the alkaline earth metals). Divalent cations of the alkaline earth metals may be used to crosslink the organic matrix to form the beads. In a particular embodiment, the crosslinking cation is provided in the form of calcium chloride.

Trace minerals provided by cations include sodium, potassium, magnesium, calcium, iron, manganese, zinc, copper, and cobalt. They can be provided in the form of soluble compounds in the form of nitrates, phosphates, hydrogen phosphates, dihydrogen phosphates, sulfates, chlorides, carbonates, bicarbonates, and molybdates. Such anions are sources of sulfur, nitrogen, phosphorus, and molybdenum, for example. Trace nutrients can also include chelators such as EDTA, including sodium and potassium salts thereof.

Microalgae nutrient packages are sold commercially or can be custom formulated. In one non-limiting example, a medium contains KH₂PO₄, CaCl₂.2H₂O, MgSO₄.7H₂O, NaNO₃, K₂HPO₄, NaCl, Na₂EDTA.2H₂O, KOH, FeSO₄.7H₂O, H₃BO₃, and a trace metal solution containing H₃BO₃, MnCl₂.4H₂O, ZnSO₄.7H₂O, Na₂MoO₄.2H₂O, CuSO₄.5H₂O, and Co(NO₃)₂.6H₂O.

In various embodiments, beads comprise from about 1 to about 3% by weight of one or more nutrients.

### Other Ingredients

The beads can contain additional additives such as enzymes, which may be incorporated to enhance inoculation or growth of the microalgae in the growth medium, or can be used as a dietary supplement for the animal that will be fed the microalgae grown from the beads. Among these are dietary enzymes such as non-starch polysaccharide-hydrolyzing enzymes such as xylanases and beta-glucanases. Other exogenous enzymes include phytases, amylases, proteases, mannanases, galactosidases, and pectinases.

### Methods of Making Beads

Beads (algae-containing pellets) containing microalgae cells, particles of an insoluble carbon source (e.g. biochar), and a crosslinked organic matrix are made by gelling an organic polymer (for example by crosslinking with a divalent cation like Ca+2) in the presence of suspended microalgae cells and the carbon source. In general, an algal char suspension is made comprising algae, polymer and insoluble carbon; then a bead volume of the suspension is contacted with a solution containing a crosslinker to gel the polymer and form the bead. Thus, in various embodiments, methods of making beads comprise:
combining water, unicellular algae, an insoluble carbon (char), and a soluble polymer (e.g., a polymer comprising a plurality of hydroxyl groups) to make an algal char suspension,
dividing the algal char suspension into bead volume (e.g., a bead droplet); and
contacting the bead volume with a solution of cross-linking agent (e.g., divalent cations) to gel the soluble polymer and form beads comprising the algae and the insoluble carbon in essentially the size and shape of the bead volume.

The algal char suspension is made by combining water, unicellular microalgae, char, and a water soluble polymer. The algae may be obtained from a culture of algae, such as may be grown in a bioreactor. In various embodiments, the algae are produced from a bioreactor using the beads of the present technology. In various embodiments, the microalgae are obtained from a culture in its exponential growth phase.

An aliquot of the cell culture is combined with a composition containing the organic polymer in solution, insoluble carbon source (e.g., biochar) and other desired components of the beads, in suspension. For example, a clay material and algae nutrients can also be combined into the char suspension.

Bead volumes of the algal char suspension are formed. Such volumes are aliquots of the algal char suspension having the volume of algae, insoluble carbon, polymer and other components desired for the beads. In general, the bead volume will be similar to the volume of the resulting bead seed.

The bead volume may comprise a droplet of the algal char suspension. The droplet may be spherical (it being understood that such a “spherical” droplet may be truly spherical, essentially spherical, spheroidal, tear-drop shaped, or otherwise irregular depending on the specific conditions by which the droplets are formed).

The resulting algal char suspension may be contacted with a solution containing the crosslinking agent. As discussed above, in various embodiments, the crosslinking agent is calcium chloride, where the Ca⁺² reacts with and bridges two of the hydroxyl groups on the soluble polymer. The contacting may be by any suitable method for contacting bead volumes to the crosslinking solution, preferably so as to maintain the discrete nature of the bead volumes. For example, bead volumes may be introduced in a droplet-by-droplet manner, in stream of discrete droplets. This instantly gels the algal char suspension of the bead volumes before it can be dispersed in the crosslinking agent solution.

One way of dividing the algal char suspension into bead volumes (e.g., globules) is to flow the suspension through a conduit, where a distal end of the conduit contains a constriction that causes individual droplets to form upon exit. The droplets then fall by gravity into (or they are delivered under the surface of) the crosslinking solution. A way of visualizing the process is to imagine the algal char suspension in an eye dropper disposed above the surface of the crosslinker solution. An operator squeezes the bulb of the dropper so as to form drops that fall into the solution below. The drops gel instantly on contact with the crosslinking solution and form essentially spheroidal beads that contain microalgae cells, char, and a crosslinked organic matrix.

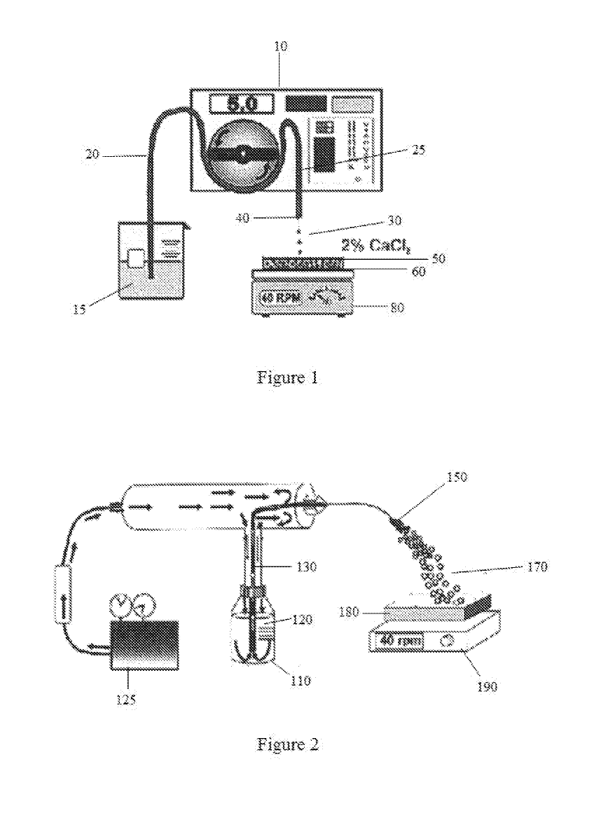

The process can be automated for industrial production. Two ways are exemplified in FIGS. 1 and 2. In FIG. 1, a peristaltic pump 10 continuously advances a suspension 15 containing algae and char through a conduit (shown as tubing 20), forming droplets 30 at a distal end 40 of the conduit 20 that fall into or are delivered beneath the surface 50 of a solution 60 containing a crosslinking agent such as calcium chloride, that is stirred with a magnetic stirrer 80 or similar device. In FIG. 2, a vessel 110 containing a suspension 120 containing algae and char is pressurized using pump 125, forcing the suspension continuously through a conduit 130 that leads from the vessel 120 to a distal end 150 of the conduit 130 equipped with a restriction that induces formation of droplets 170 that are delivered into a crosslinking solution such as calcium chloride 180, that is stirred with a magnetic stirrer 190 or similar device.

In any of the methods described here, the size of the bead produced depends on the size of the droplets exiting from the conduit. The size depends on the speed at which the suspension flows through the conduit, the shape and dimensions of the restriction at the distal end, the viscosity of the char suspension, and other factors.

In some continuous processes, such as shown in FIGS. 1 and 2, crosslinked beads are formed that have essentially the same or similar shape, size, and density. However, in various embodiments, it is desirable to have a collection of beads of differing densities, or having what is called a diversity of densities so to vary from the specific gravity of water or of the cellular growth medium in which the beads are to be inoculated. Accordingly, the present technology provides bead seed mixtures comprising beads having a plurality of densities (or specific gravities), such as population of beads having a range of densities. In various embodiments, the specific gravity of the beads may range from −10% to +10% of the specific gravity of water, or of the culture media in which the beads are used in methods of the present technology.

Without limiting the scope, mechanism, function or utility of present technology, it is believed that bead mixtures containing beads having a diversity of densities will disperse better into an algal growth medium when the medium is inoculated with the beads, relative to a quantity of beads having uniform densities. That is, in a composition with a diversity of densities, beads with the same density as the growth medium will tend to be in the middle of the medium, while heavier (more dense) beads will tend to be near the bottom and lighter (less dense) beads will tend to rise and be toward the top of the growth medium, as that growth medium is agitated in contact with the beads for inoculation.

One way to make a composition of beads having a diversity of densities is to make separate batches of seeds, each of more or less uniform density, and combine them together. To illustrate, this process would involve making a first batch of beads with a first density, a second batch with a second density, and optionally a third batch with a third density, a fourth batch with a fourth density, and so on. The density of the batches can be varied in a number of ways, such as by varying the amount of char suspended in the char suspension prior to crosslinking, or by varying the amount of the water soluble polymer in the char suspension.

Another way to make a composition with a diversity of densities is to take advantage of gravity to create a gradient of densities in the conduit before the char suspension exits from the conduit in the form of droplets. Referring again to FIG. 1, a conduit 20 contains a section with a vertical component 25. The char suspension 15 is advanced through the conduit 20 until it reaches the restriction at the distal end 40 and the char suspension fills the vertical component 25. Advance of the circulating char suspension is temporarily halted such as by momentarily turning off the pump 10. As a result, the char suspension stays momentarily in the vertical component 25 and does not immediately exit in the form of droplets. During the momentary halt, the char suspension tends to settle in the vertical component 25. This forms a density gradient in the vertical component, with the bottom of the component having the most char and therefore the highest density. At the same time, the char suspension at the top of the vertical component is depleted of char, and has the lowest density. If then the advancement through the conduit is restarted (such as by re-engaging the pump), the column with a gradient of densities (highest to lowest from top to bottom) is emptied as a series of droplets into the crosslinking solution. The crosslinked beads thus formed have a variety of densities corresponding to the density gradient formed in the conduit. In this way a composition containing beads with a diversity of densities is produced continuously.

Another way of obtaining a collection of beads having a diversity of densities is to use a gas (e.g., nitrogen, carbon dioxide or air) to ballast the beads as they are formed. In a representative embodiment, a bubbling stone is used to release carbon dioxide (CO2) into the char suspension before it is to be contacted with the solution containing the divalent cation crosslinking agent. As the char suspension is delivered into the crosslinking solution, it is almost instantaneously gelled by reaction of divalent cations in the crosslinking solution with the soluble polymer having a plurality of hydroxyl groups. Gelation of the char suspension forms spherical gelled particles that contain some entrapped CO2 as well as cells of the microorganism, char, and optional aluminosilicate. The CO2 bubbles are statistically distributed in the gelled spheres, with some having no bubbles, others having 1 bubble, and still others having A 2 bubbles or more. The more CO2, the lower will be the density of the bead seed. The result of the process is that a composition is formed that contains beads of varying densities. As the CO2 is entrapped in a crosslinked matrix, it tends to remain in the beads during further processing so that the beads inoculated into the algae growth medium will have different densities.

In various embodiments, upon formation when the droplets of char suspension contact the crosslinking solution, the beads are spheroidal (as defined above). If desired, a batch of beads can be made where all the beads are of the same size. After contact, the beads are stirred with the crosslinking solution for a minute or two (longer does not hurt) to harden them sufficiently for further handling. The beads can also be rinsed with distilled or deionized water.

If desired, the beads can be molded into other shapes after hardening just described. In various embodiments, the beads are molded to have at least one irregular surface feature, such as surface region that is flat, concave or convex, as discussed above. For example, the spheroidal beads can be molded into a truncated icosahedron comprising a plurality of pentagonal and hexagonal panels. In one embodiment a spherical polyhedron is formed by configuring each panel to form a convex surface. In a particular embodiment, at least one panel of the bead seed is configured to have a concave surface, so that currents and ricochet forces do not result in a regular or repeating angle of deflection off of surfaces. These aspects of the shape of the beads are presented in U.S. Pat. No. 9,243,219, Dimitrelos, issued Sep. 19, 2013, the disclosure of which is useful for background information and is hereby incorporated by reference.

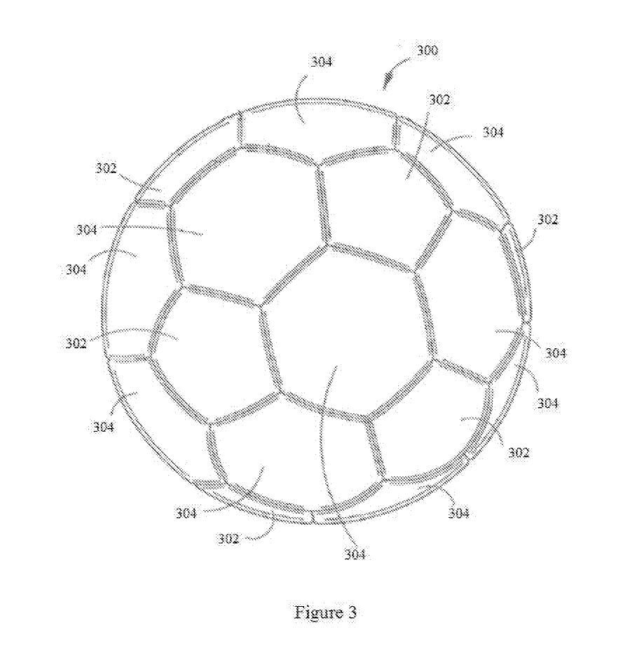

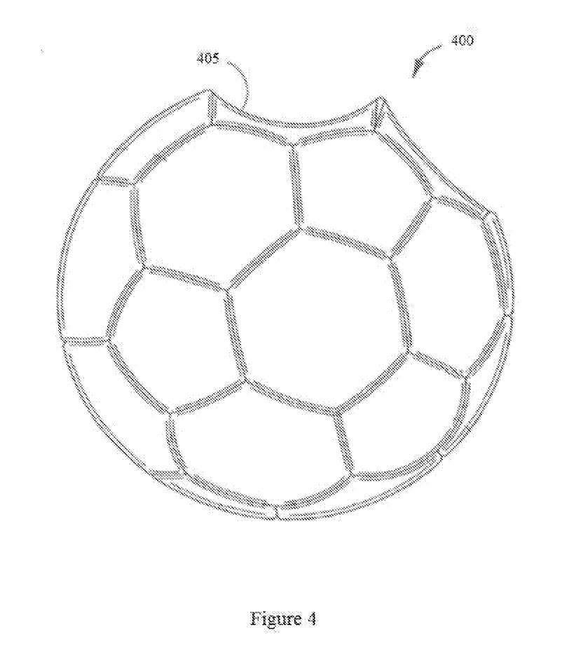

Referring to FIG. 3, in some embodiments, the beads 300 are formed into a truncated icosahedron comprising a plurality of pentagonal 302 and hexagonal 304 panels. In particular, each pentagonal panel 302 is bordered by five hexagonal panels 304. In one embodiment a spherical polyhedron is formed by configuring each panel to form a convex surface. This causes the beads to spin through the water to mix and disrupt the boundary layer effect of the water on the alga.

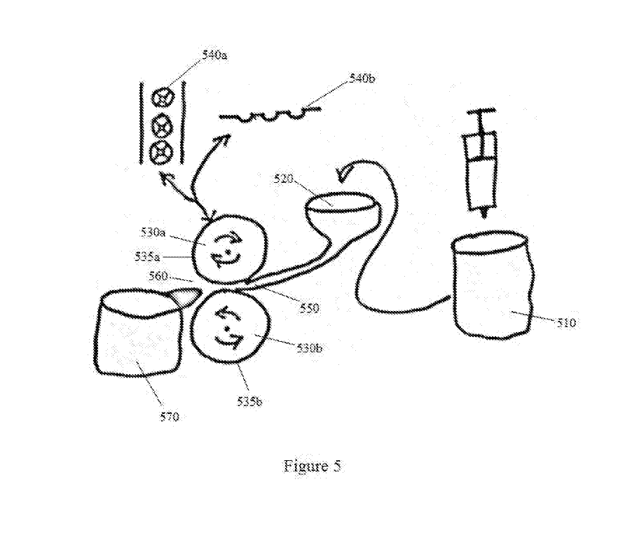

FIG. 4 illustrates yet another embodiment of a bead 400 wherein at least one panel of the bead 400 is configured to have a concave surface 405 so that currents and ricochet forces do not result in a regular or repeating angle of deflection off of surfaces. This is so that the beads will spin through the water to mix and disrupt the boundary layer effect of the water on the alga.

In some embodiments, it is therefore desirable to produce a batch of beads of uniform size, for ease of molding or other post processing. In an exemplary method depicted in FIG. 5, a collection of beads 510 is collected in a hopper 520, from whence they are fed one by one into two rotating drums 530a, 530b the circumferential faces 535a, 535b of which contain meshing molds in the shape of a docosahedron or other desired shape, illustrated in top view 540a and side view 540b. The raw beads are fed from the hopper 520 into the entrance 550 of the mold and proceed to the outlet 560 of the mold 530, where they are collected in container 570. Conventional tablet presses can be adapted to carry out molding steps on the beads.

Following production, the beads may be used directly in production of algae, or may be stored for subsequent use. For example, the beads may be stored in insulated containers at low temperatures (e.g., about 50° F. or lower). This keeps the microorganisms within the beads in suspended animation and allows for long term storage. Without limiting the scope or function of the present technology, reduced temperatures may also allow for hardening of the crosslinked bead, as the cold storage may help in the contraction of the bead seed's cros slinking matrix. The beads are preferably kept dry and in the dark. Under these conditions, the beads can be transported and shipped to customer, as they are not perishable.

### Methods of Producing Algae Using Beads

The present technology provides methods for producing algae using beads, as described above. Such methods generally comprise contacting a plurality of beads with growth medium. In various embodiments, algae grow within the seeds and are released into the medium. Without limiting the scope, mechanism, function or utility of present technology, methods comprising the growth of algae using beads may, in some embodiments, accelerate growth of algae relative to conventional methods, offering such benefits as decreased time to exponential growth phase and increased alga culture stability (i e , minimizing or eliminating the incidence of culture crashing that can occur with conventional methods).

Before inoculation, the beads can be kept in cold storage at a temperature of about 50° F. or less (e.g., about 45° F.). Before inoculation into a vessel, the beads may be partially tempered by leaving them out at room temperature. Otherwise going too fast from cold to warm may cause cell shock, leading to cell retardation or even cell death. Next, the almost fully tempered beads are added to the warm water environment which helps further expand the concentrated crosslinking matrix plus the warm water environment awakens the algae biological functions.

For inoculation, the beads are placed in a vessel containing a growth medium like that in which the cells inside the beads were originally grown. As a rule of thumb, 10 to 15 unit volumes of beads are added to 100 unit volumes of growth medium (10%-15% v/v), for example 10-15 liters of beads in a 100 liter vessel. Variations are possible depending on the nature of the microorganism, the cell count in the beads, and the level of nutrients and light supplied in the vessel.

When the microorganisms are algae species, the vessel acts as a photobioreactor, where the microbes are provided with light photons to support photosynthesis. The beads are set in motion relative to the growth medium—by rocking, stirring, and so on—and the motion causes the algae cells in the beads to escape, inoculating the medium. The contents of the beads provide nutrients during a first phase of growth, analogous to the function of a seed, which provides nutrients for a plant just starting out. After inoculation, algae growth is accomplished by replenishing the nutrients in the vessel as needed, and by providing microalgae in the vessel with visible light and with carbon dioxide to guide photosynthesis. General methods for culturing algae are described in U.S. Pat. No. 9,243,219, Dimitrelos, issued Sep. 19, 2013, the disclosure of which hereby incorporated by reference.

Embodiments of the present technology are further illustrated through the following non-limiting examples.

### EXAMPLES

### Example 1

Beads may be made according to the following general procedure, described on a lab scale using microalgae. Other microorganisms can be used by substituting them for the microalgae. Portions can be automated and scaled according to known methods including those illustrated in FIGS. 1 and 2.
1. Grow a microalga strain and concentrate active algal culture as it is in the middle exponential phase.
2. Prepare solutions to make alginate beads:
   Dissolve 3 g of sodium alginate in 100 ml of cold, pure water. Stir every half hour or so and leave overnight, stir in the morning.
   Dissolve 4 g of calcium chloride in 200 ml of pure water in a 500 ml beaker.
3. Make alginate beads:
   When making up the alginate or diluting the algal culture it is preferred to use pure water, i.e. deionized or distilled; otherwise calcium ions in the water will cause the alginate to ‘set’ prematurely.
   Different concentrations of sodium alginate (around 1.5%-3%) can be used to create beads having different wall thickness and different density related to the amount of alginate in the solution. Sufficient alginate (or other soluble polymer having a plurality of hydroxyl groups) is added to prepare a char suspension as a viscous mixture that will drip steadily through a syringe. The different concentrations create the wall thickness so some walls are thinner than others. This varying thickness keeps some beads sealed longer for mixing than others and the thinner ones are more fragile so the algae are released into the water for inoculation. This allows for the specific gravity of each bead to vary in order to float, bob and sink. Since algae are everywhere in the water, the beads need to be in many planes so the beads disrupt the boundary layer associated with alga.

### Preparation of Algae Beads

4. Mix 5 cm³ of the algal culture with 5 cm³ of the 3% sodium alginate solution in a beaker. Add desired levels of biochar, and clay to make a char suspension.
5. Clamp a syringe barrel above a beaker of calcium chloride solution making sure the tip of the syringe is well above the solution in the beaker.
6. Pour the alga/alginate mixture through the syringe barrel so it drips through and forms beads in the beaker. Swirl the beaker gently as the drops fall.
7. Allow the beads to harden for a few minutes before straining them out of the beaker through a tea strainer, then rinse the beads in distilled water. The algae in the beads will stay alive for several months in a stoppered bottle in the refrigerator.

### Example 2

### Components and Methods for Creating the Algae Beads

First, 6 g of dry base components comprising of about 70% by weight of sodium alginate, 15% by weight of biochar and about 15% by weight of kaolin clay are added to 150 ml of pure water. The solution is mixed every half hour or so until alginate is fully dissolved and then added to equal parts of a microalgae culture growing in exponential mode in the presence of a nutrient broth like Bold's Basal Medium (BBM). This makes a char suspension.

Next, the BBM/dry base component homogenous solution is entrapped to create the algae bead-seed (see also Example 1). The entrapment begins by placing the homogenous BBM Mixture/dry base component homogenous solution in a vessel or conduit having a small orifice (such as a syringe tip) at a distal end. That solution is dripped from the syringe or orifice. The rate of dripping can vary depending on a variety of parameters. The solution is dripped into a co-immobilizing solution of calcium chloride. As the BBM Mixture/dry base component homogenous solution (i.e. the char suspension) drips from the conduit through the syringe tip into a co-immobilizing solution of calcium chloride, malleable seeds inside the solution are formed. These seeds can be pressed into a desired shape to form cross-linked/hardened “beads”, which can be rinsed with deionized or distilled water and finally stored in the refrigerator in airtight containers.

### Exemplary Embodiments

The following is a non-limiting list of exemplary embodiments.

1. An algal bead comprising unicellular microalgae, char, and a crosslinked organic matrix.

2. The bead of embodiment 1, further comprising a clay.

3. The bead of embodiment 1, wherein the organic matrix before crosslinking comprises a plurality of hydroxyl groups that react with a divalent cation to crosslink the matrix.

4. The bead of embodiment 3, wherein the organic matrix comprises a water soluble polysaccharide.

5. The bead of embodiment 4, wherein the water soluble saccharide comprises an alginate salt.

6. The bead of embodiment 4, wherein the water soluble saccharide comprises a galactomannan.

7. The bead of embodiment 4, wherein the water soluble saccharide comprises a gellan gum.

8. The bead of embodiment 4, wherein the water soluble saccharide comprises carrageenan.

9. The bead of embodiment 4, wherein the water soluble saccharide comprises agarose.

10. The bead of embodiment 2, wherein the clay comprises kaolin.

11. The bead of embodiment 1, wherein the char comprises a biochar.

12. The bead of embodiment 11, wherein the biochar is produced from algal biomass.

13. The bead of embodiment 11, wherein the biochar is produced from rice hulls.

14. The bead of embodiment 1, further comprising soluble salts of cations selected from Na+, K+, Mg+2, Ca+2, Fe+3, Mn+2, Zn+2, Cu+2, and Co+2.

15. The bead of embodiment 14, comprising soluble salts of one or more anions selected from nitrates, phosphates, hydrogen phosphates, dihydrogen phosphates, sulfates, chlorides, EDTA, carbonates, bicarbonates, and molybdates.

16. The bead of embodiment 1, wherein the microalgae are selected from the group consisting of Chlorella, Spirulina, Dunaliella, Haematococcus, Crypthecodinium, Schizochytrium, Scenedesmus, Aphanizomenon, Arthrospira, Odontella, Isochrysis, Nannochloropsis, Tetraselmis, Phaeodactylum, Porphyridium, and combinations thereof.

17. The bead of embodiment 3, wherein the divalent ion is selected from Be+2, Mg+2, Ca+2, Sr+2, and Ba+2.

18. A method of making a bead, comprising:
Combining water, unicellular algae, a char, and a soluble polymer to make a char suspension, wherein the polymer comprises a plurality of hydroxyl groups that can react with divalent cations to crosslink,
Dividing the char suspension into bead shaped globules; and
Contacting the globules with a solution of divalent cation to gel the soluble polymer and form beads comprising algae and char.

19. The method of embodiment 18, wherein the divalent cation is selected from Be+2, Mg+2, Ca+2, Sr+2, and Ba+2.

20. The method of embodiment 18, wherein the soluble polymer is selected from polyvinyl alcohol, alginate, agarose, carrageenan, galactomannans, gellan gum, and guar gum.

21. The method of embodiment 18, wherein the char comprises a biochar.

22. The method of embodiment 18, wherein the biochar is produced from algal biomass.

23. The method of embodiment 18, wherein the biochar is produced from rice hulls.

24. The method of embodiment 18, wherein the char suspension further comprises a clay.

25. The method of embodiment 18, wherein the char suspension further comprises any additive on the list of components generally recognized as safe.

26. The method of embodiment 18, wherein dividing the char suspension into bead shaped globules comprises flowing the char suspension in a conduit from a proximal to a distal end, wherein the distal end of the conduit comprises a restriction that causes droplets to form upon exit from the conduit.

27. The method of embodiment 26, wherein the conduit comprises at least one vertical passage before the exit on the distal end, and the beads formed from the droplets exhibit a diversity of densities.

28. A method of culturing unicellular algae comprising contacting the bead of any of embodiments 1 to 17 with an algae culture medium under conditions where the algae grow in the beads and then escape the bead into the medium.

29. The method of embodiment 28, comprising placing the beads in motion relative to the medium.

30. The method of embodiment 29, comprising stirring the medium while the medium is in contact with the beads.

31. The method of embodiment 29, comprising rocking a vessel that contains the beads and the medium.

32. A method of culturing unicellular algae comprising contacting a bead made by the method of any of embodiments 18 to 27 with an algae culture medium under conditions where the algae grow in the beads and then escape the bead into the medium.

33. The method of embodiment 28, comprising placing the beads in motion relative to the medium.

34. The method of embodiment 29, comprising stirring the medium while the medium is in contact with the beads.

35. The method of embodiment 29, comprising rocking a vessel that contains the beads and the medium.

36. A method for growing algae in cell culture, comprising
contacting an aqueous growth medium in a vessel with an algae bead; and
agitating the growth medium while in contact with the bead;
wherein the algae bead comprises algae cells, a (crosslinked) organic matrix, and a carbon based nutrient.

### Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. For example, a component which may be A, B, C, D or E, or combinations thereof, may also be defined, in some embodiments, to be A, B, C, or combinations thereof. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

“A” and “an” as used herein indicate “at least one” of the item is present; a plurality of such items may be present, when possible.

Numeric values stated herein should be understood to be approximate, and interpreted to be about the stated value, whether or not the value is modified using the word “about.” Thus, for example, a statement that a parameter may have value “of X” should be interpreted to mean that the parameter may have a value of “about X.” “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Further, the phrase “from about A to about B” includes variations in the values of A and B, which may be slightly less than A and slightly greater than B; the phrase may be read be “about A, from A to B, and about B.” Disclosure of values and ranges of values for specific parameters (such as temperatures, molecular weights, weight percentages, etc.) are not exclusive of other values and ranges of values useful herein.

It is also envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3,3-10, and 3-9.

What is claimed is:

1. An algal bead comprising unicellular microalgae, char, and a crosslinked organic matrix.

2. The bead of claim 1, further comprising a clay.

3. The bead of claim 1, wherein the organic matrix before crosslinking comprises a plurality of hydroxyl groups that react with a divalent cation to crosslink the matrix.

4. The bead of claim 3, wherein the organic matrix comprises a water soluble polysaccharide selected from the group consisting of alginate salts, galactomannan, gellan gum, carrageenan, agarose, and mixtures thereof.

5. The bead of claim 2, wherein the clay comprises kaolin.

6. The bead of claim 1, wherein the char comprises a biochar.

7. The bead of claim 6, wherein the biochar is produced from algal biomass or rice hulls.

8. The bead of claim 1, further comprising soluble salts of cations selected from Na+, K+, Mg+2, Ca+2, Fe+3, Mn+2, Zn+2, Cu+2, and Co+2.

9. The bead of claim 8, comprising soluble salts of one or more anions selected from nitrates, phosphates, hydrogen phosphates, dihydrogen phosphates, sulfates, chlorides, EDTA, carbonates, bicarbonates, and molybdates.

10. The bead of claim 1, wherein the microalgae are selected from the group consisting of Chlorella, Spirulina, Dunaliella, Haematococcus, Crypthecodinium, Schizochytrium, Scenedesmus, Aphanizomenon, Arthrospira, Odontella, Isochrysis, Nannochloropsis, Tetraselmis, Phaeodactylum, Porphyridium, and combinations thereof.

11. The bead of claim 3, wherein the divalent ion is selected from Be+2, Mg+2, Ca+2, Sr+2, and Ba+2.

12. A method of making a bead, comprising:
Combining water, unicellular algae, a char, and a soluble polymer to make a char suspension, wherein the polymer comprises a plurality of hydroxyl groups that can react with divalent cations to crosslink,
Dividing the char suspension into bead shaped globules; and
Contacting the globules with a solution of divalent cation to gel the soluble polymer and form beads comprising algae and char.

13. The method of claim 12, wherein the divalent cation is selected from Be+2, Mg+2, Ca+2, Sr+2, and Ba+2.

14. The method of claim 12, wherein the soluble polymer is selected from polyvinyl alcohol, alginate, agarose, carrageenan, galactomannans, gellan gum, and guar gum.

15. The method of claim 12, wherein the char comprises a biochar produced from algal biomass or rice hulls.

16. The method of claim 12, wherein the char suspension further comprises a clay.

17. The method of claim 12, wherein dividing the char suspension into bead shaped globules comprises flowing the char suspension in a conduit from a proximal to a distal end, wherein the distal end of the conduit comprises a restriction that causes droplets to form upon exit from the conduit.

18. A method of culturing unicellular algae comprising contacting the bead of claim 1 with an algae culture medium under conditions where the algae grow in the beads and then escape the bead into the medium.

19. The method of claim 18, comprising placing the beads in motion relative to the medium.

20. A method for growing algae in cell culture, comprising
contacting an aqueous growth medium in a vessel with an algae bead; and
agitating the growth medium while in contact with the bead;
wherein the algae bead comprises algae cells, a (crosslinked) organic matrix, and a carbon based nutrient.
